SEQUENCE LISTING (1 sequence)

SEQ ID NO: 1 - DNA, 33 bp, Artificial Sequence
  Synthetic Probe for GBS cfb
acacctcaacagtgaactagtcgtacatgaaga

(19) United States
(12) Patent Application Publication
(10) Pub. No.: US 2006/0199187 A1
(43) Pub. Date: Sep. 7, 2006

(54) Enzyme amplified electrochemical DNA detection

(76) Inventors: Mark E. Meyerhoff, Ann Arbor, MI (US); Hyoungsik Yim, Ann Arbor, MI (US); Youngmi Lee, Knoxville, TN (US); John S. Althaus, Saline, MI (US); Lara M. Wheeler, Adrian, MI (US); Kyong-Hoon Lee, Ann Arbor, MI (US)

Correspondence Address: JULIA CHURCH DIERKER;DIERKER & ASSOCIATES, P.C., 3331 W. BIG BEAVER RD. SUITE 109, TROY, MI, 48084-2813, US

(21) Appl. No.: 11/070,570

(22) Filed: Mar. 2, 2005

(51) Int. Cl. C12Q 1/68; C07H 21/04

(52) U.S. Cl. 435/6; 536/24.3

(57) ABSTRACT

An oligonucleotide probe for DNA detection is disclosed. The oligonucleotide probe includes a site-specific sequence. Either a co-factor or a mediator is conjugated to the site-specific sequence. The co-factor is adapted to produce an enzymatic signal that is electrochemically detectable. The mediator is adapted to enhance an enzymatic signal that is electrochemically detectable.

### BACKGROUND

The present disclosure relates generally to DNA detection, and more particularly to the electrochemical detection of DNA using a specially designed oligonucleotide probe.

Genetic testing and assays have the potential for use in a variety of applications, ranging from genetic diagnostics of human diseases to detection of trace levels of pathogens in food products. Currently, more than 400 diseases can be diagnosed by molecular biology analysis of nucleic acid sequences. It is likely that additional tests will be developed as further genetic information becomes available. DNA diagnostic devices enable clinicians to efficiently detect the presence of a whole array of genetically based diseases, including, for example, AIDS, Alzheimer's, and various forms of cancer.

DNA amplification processes are currently used for real-time and end-point detection of specific DNA sequences. The current chemistries allow detection of PCR products via the generation of a fluorescent signal. Generally, the required reagents for these systems are expensive to synthesize, and in some instances, the systems require use of expensive fluorescence instrumentation for detection. Some techniques include binding dye to a double stranded DNA sequence and thus do not use a probe designed for any particular target being analyzed. However, detection of PCR amplified DNA by such a method requires extensive optimization since the dye cannot distinguish between specific and non-specific products accumulated during PCR. With this type of technique, follow-up assays are used, in some instances, to validate obtained results.

The rising use of DNA diagnostic testing devices has produced a need for low-cost, highly portable DNA detection devices (for example, a glucometer-type “lab-on-a-chip” device) for use in various markets including health care, agriculture, food testing and bio-defense. Generally, it would be desirable that any new DNA diagnostic devices integrate several functional analysis components within the same platform. Further, it would be desirable that such devices be reliable, inexpensive, and able to simplify the detection of target DNA.

### SUMMARY

An oligonucleotide probe for DNA detection is disclosed. The oligonucleotide probe includes a site specific sequence. Either a co-factor or a mediator is conjugated to the site specific sequence. The co-factor is adapted to produce an enzymatic signal that is electrochemically detectable. The mediator is adapted to enhance an enzymatic signal that is electrochemically detectable.

A method of detecting target DNA in a sample is also disclosed. The method includes performing a DNA amplification process on the sample. An oligonucleotide probe, including a site-specific sequence and either a co-factor or a mediator conjugated to the site-specific sequence, is exposed to exonuclease activity. This exposure releases a probe fragment which includes the co-factor or the mediator. The probe fragment is then combined with an apo-enzyme or a holo-enzyme. Combining the probe fragment having the co-factor with the apo-enzyme produces an enzymatic signal that is electrochemically detectable; and combining the probe fragment having the mediator with the holo-enzyme enhances an enzymatic signal that is electrochemically detectable. The enzymatic signal, which is electrochemically detectable, confers detection of the target DNA.

### BRIEF DESCRIPTION OF THE DRAWINGS

Objects, features and advantages of embodiments of the present invention will become apparent by reference to the following detailed description and drawings, in which:

FIG. 1 is a schematic flow diagram illustrating an embodiment of making an embodiment of an oligonucleotide probe;

FIG. 2 is a schematic view of an embodiment of a method of detecting DNA;

FIG. 3 is an exploded, schematic view of a specific example embodiment of the method depicted in FIG. 2;

FIG. 4 is a schematic view of a specific example embodiment of the method depicted in FIG. 2, including a barrier membrane and a reagent;

FIG. 5 is a schematic view of an alternate embodiment of a method of detecting DNA;

FIG. 6 is a schematic view of a specific example embodiment of the method depicted in FIG. 5; and

FIG. 7 is a graph depicting an amplified amperometric signal involving glucose dehydrogenase (GDH) activity when positive and negative PCR samples were tested; the PCR samples contained a PQQ labeled oligonucleotide probe for group beta streptococcus (GBS) gene targeting; PCR samples were collected after 50 thermal cycles.

### DETAILED DESCRIPTION

Embodiment(s) disclosed herein advantageously combine an oligonucleotide probe and the production of an enzyme amplified electrochemical detectable signal, both of which may be incorporated into a DNA diagnostic device. This combination provides an enzyme-based electrochemical method to detect DNA amplified via polymerase chain reaction (PCR) or other DNA amplification methods. It is to be understood that embodiment(s) of the probe may be integrated with, for example, a strip for end-point PCR detection or with a thermo-cycler for real-time PCR detection. Embodiment(s) of the probe and method may also be integrated with a sample concentrator and/or cell lyses components into a microfluidic and/or a macrofluidic platform. Such integration may advantageously assist in substantially reducing both the size of the device and its manufacturing cost.

In an embodiment, the miniaturization of the diagnostic device is achieved by coupling embodiment(s) of the probe with a signal amplification strategy (using apo-enzyme(s) with co-factor(s) or holo-enzyme(s) with electroactive mediator(s)) and incorporating microelectrodes/electrochemical tags in micro-size or nano-size reaction chambers, substantially without inhibiting the reaction. It is to be understood that embodiment(s) of the detection method and/or probe, with inherent enzyme signal amplification, substantially eliminates the need for expensive optical detection and the associated equipment, yet allows for DNA detection with fewer amplification cycles.

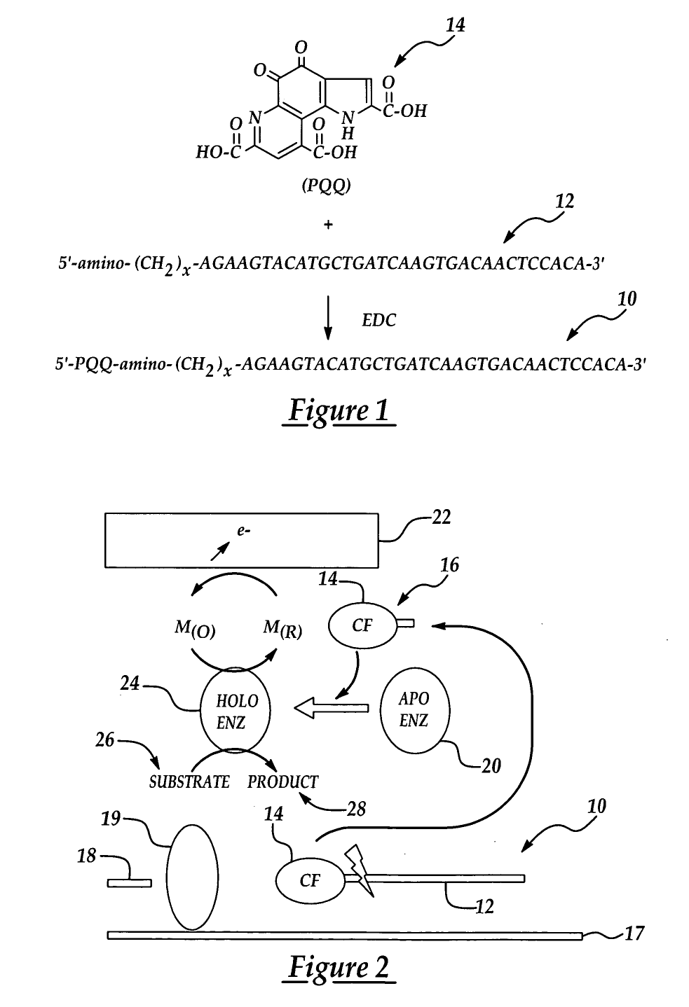

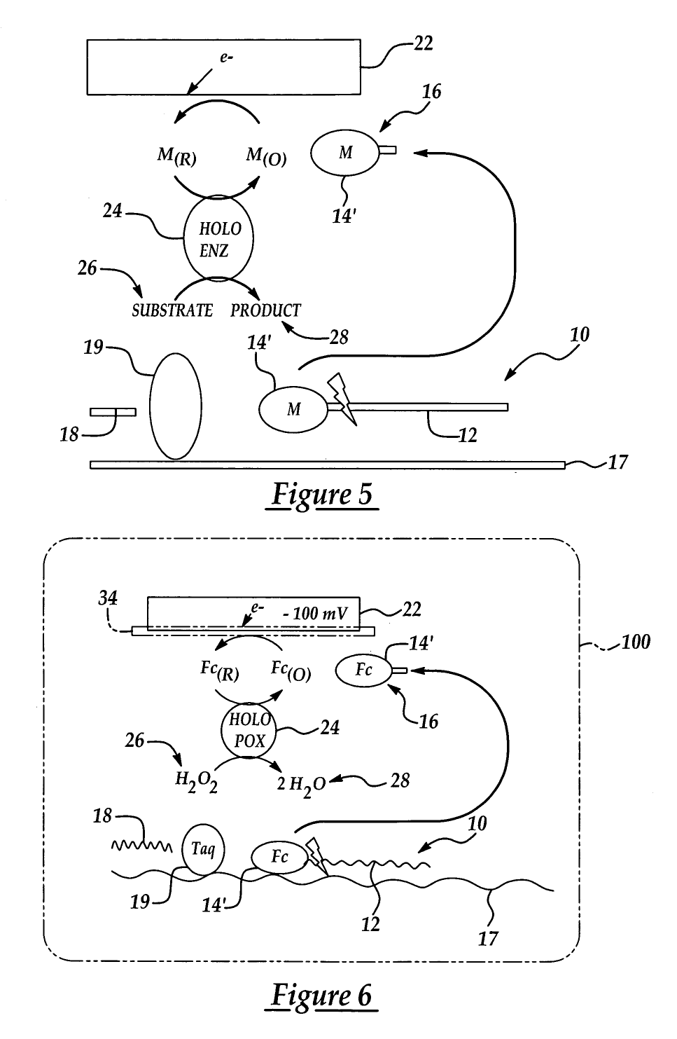

Referring now to FIG. 1, an embodiment of making an oligonucleotide probe 10 is schematically depicted. Generally, embodiments of the intact oligonucleotide probe 10 include a site-specific sequence 12 labeled with a co-factor (CF) 14 (e.g. co-enzyme or prosthetic group) (shown in FIGS. 2-4) or a mediator (M) 14′ (shown in FIGS. 5-6). The co-factor (CF) 14 or the mediator (M) 14′ may be conjugated at any spot along the site-specific sequence 12. In non-limitative example embodiments, the co-factor (CF) 14 or the mediator (M) 14′ may be attached to the 5′ end, the 3′ end, and/or anywhere between the two ends, as desired.

In a non-limitative example, the co-factor (CF) 14, such as PQQ shown in FIG. 1, may be conjugated at the 5′ end of the site specific sequence by adding 1-(3-dimethylaminopropyl)-3-ethylcarbodiimide (EDC or EDAC; 1 mg/ml) into a PQQ solution. After reacting the PQQ with EDAC at about 4° C., N-hydroxysuccinimide (NHS; 0.6 mg/ml) is injected into the solution and shaken. This results in the modification of the carboxyl groups and the formation of amine-reactive NHS esters. A solution of the desired 5′amine-derivatized oligonucleotide may be added to the activated PQQ and allowed to react. In an example embodiment, the oligonucleotide solution contains a 5′amine and a carbon linker (e.g. (CH₂)ₓ).

In embodiment(s) of the method, the co-factor (CF) 14 is adapted to produce an enzymatic signal that is electrochemically detectable. As used herein, the term “produce” means indirectly or directly generating the enzymatic signal. In a non-limitative example, indirectly producing includes binding the co-factor (CF) 14 to an enzyme to form an activated enzyme that is capable of catalyzing a reaction that results in an electrochemically detectable enzymatic signal. In alternate embodiment(s) of the method, the mediator (M) 14′ is adapted to enhance an enzymatic signal that is electrochemically detectable.

Generally, as described in more detail hereinbelow, co-factor (CF) 14 is used in an embodiment where the co-factor (CF) 14 portion of the probe 10 binds with an apo-enzyme; while the mediator (M) 14′ is used in an embodiment where the mediator (M) 14′ portion of the probe 10 is put in contact with a holo-enzyme to transfer electrons from the active site of the holo-enzyme to an electrode. Non-limitative examples of the co-factor (CF) 14 include prosthetic groups (organic and covalently bound to an enzyme), co-enzymes (organic and non-covalently bound to an enzyme), and metal-ion activators. Specific non-limitative examples of co-factors include pyrroloquinoline quinine (PQQ), flavin adenine dinucleotide (FAD), nicotinamide adenine dinucleotide phosphate (NADP), heme, and the like. Non-limitative examples of the mediator 14′ include ferrocene, ferrocene derivatives, dichlorophenol indolphenol (DCIP), ubiquinone (Q₀), Ru(III) complexes, Os(III) complexes, phenazoinum methosulfate (PMS), and the like. Non-limitative examples of metal ion activators include iron, copper, manganese, magnesium, zinc, and the like, and combinations thereof.

The non-limitative example shown in FIG. 1 depicts pyrroloquinoline quinone (PQQ) as the co-factor (CF) 14, and 5′-amino-hexyl-AGAAGTACATGCTGATCAAGTGACAACTCCACA-3′ as the site-specific sequence 12. The PQQ is conjugated to the amino group of the sequence 12. The non-limitative example oligonucleotide probe 10 shown in FIG. 1 is capable of detecting target DNA from group beta streptococcus (GBS). It is to be understood, however, that the probe 10 may be made complimentary to any target DNA. Further, the probe 10 may be designed to hybridize or anneal to its complementary single strand DNA sequence within an amplicon domain defined by a pair of primer oligonucleotides or between forward and reverse primers.

Referring now to FIG. 2, an embodiment of the method of detecting target DNA in a sample using an embodiment of the probe 10 is schematically depicted. Embodiment(s) of the method integrate DNA amplification processes (non-limitative examples of which include real-time and end-point PCR) with enzymatic signal amplification. More specifically, PCR-dependent exonuclease activity can trigger enzymatic generation or amplification of a measurable electrochemical enzymatic signal, a non-limitative example of which is current. The enzymatic signal(s) may be measured via any suitable technique, non-limitative examples of which include voltammetry, amperometry, coulometry, potentiometry, conductivity, and/or the like, and/or combinations thereof.

Embodiment(s) of the method generally include performing a DNA amplification process on a sample 17, exposing an embodiment of the probe 10 to exonuclease activity, either combining a prosthetic group probe fragment to an apo-enzyme or a mediator probe fragment to a holo-enzyme, and electrochemically detecting an enzymatic signal that results from the combination of the prosthetic group with the apo-enzyme or the contact of the mediator to the holo-enzyme. It is to be understood that these steps may be performed substantially simultaneously or sequentially.

Non-limitative examples of the DNA amplification processes include end-point PCR, real-time PCR, and RCA. Either PCR process may include a PCR mixture and/or sample formulated such that it is compatible with desired chemistries for enzymatic signal amplification and electrochemical detection. Such a formulation may include, but is not limited to the following: substrate(s) (a non-limitative example of which is glucose), probes 10, buffers (non-limitative examples of which include Tris, HEPES, phosphate, and the like), mediators (non-limitative examples of which include ferrocene derivatives, PMS, Q₀, DCIP, Ru(III) complexes, and the like), stabilizers (non-limitative examples of which include CaCl₂, MgCl₂, and the like), enzyme thermal stabilizers, barriers, oligo binders, and/or mixtures thereof.

As depicted in FIG. 2, the intact probe 10 includes the sequence 12 having a co-enzyme as the co-factor (CF) 14 conjugated thereto. During the DNA amplification process, 5′→3′ exonuclease activity of a DNA polymerase enzyme 19 results in the hydrolysis of the probe 10. The size and locations of DNA being amplified (amplicon) is determined, at least in part, by a pair of primer oligonucleotides (arbitrarily designated forward and reverse primers) which are complimentary and hybridize specifically to double stranded template DNA that has been denatured. As depicted, forward primer 18 is not attached to the DNA polymerase enzyme 19. It is to be understood that the forward primer 18 in the PCR mix hybridizes to a complementary region on the target DNA 17. The DNA polymerase enzyme 19 has a binding site that recognizes this structure. In this case, the target DNA 17 which hybridizes to the primer 18 is a double stranded structure that contains a 3′ terminus provided by the primer 18. The DNA polymerase enzyme 19 then extends the primer 18 by filling in complementary bases over the single stranded template.

It is to be understood that the DNA polymerase 19 may be any suitable polymerase, including, but not limited to Taq DNA polymerase (examples of which include native, recombinant, and with dNTPs), thermostable polymerases, high fidelity polymerase (with dNTPs), Pfu DNA polymerase, Bst polymerase, Tfl polymerase, Tgo polymerase, Tth polymerase, and the like, and combinations thereof.

The sequence of primer 18 is based on the sequence of the target DNA 17. Non-limitative examples of specific primers 18 include bacterial PCR primers, bovine PCR primers, canine cytokine primers, canine PCR primers, heat shock gene primers, human PCR primers, mouse PCR primers, porcine primers, porcine cytokine primers, rabbit cytokine PCR primers, rat PCR primers, viral PCR primers, and yeast PCR primers.

Examples of bacterial PCR primers include, but are not limited to C. pneumonia 16S rRNA PCR primers, C. trachomatis PCR primers, E. coli PCR primers, H. pylori PCR primers, M. tuberculosis PCR primers, mycoplasma PCR primers, N. gonorrhoea PCR primers, and the like.

Non-limitative examples of bovine PCR primers include bovine chemokine primers, bovine cytokine PCR primers, bovine growth factor BP PCR primers, bovine growth factor PCR primers, bovine growth hormone PCR primers, and the like.

Specific examples of human PCR primers include, but are not limited to human angiopoietin PCR primers, human apoptic gene primers, human bcr-ABL primers, human beta-thalassemia PCR primers, human cancer related PCR primers, human caspase PCR primers, human CD gene PCR primers, human cell cycle PCR primers, human chemokine primers, human cyclogenase PCR primers, human cytokine gene primers, human DCC PCR primers, human DMD PCR primers, human ECM PCR primers, human growth factor BP PCR primers, human growth factor primers, human housekeeping gene primers, human kinase PCR primers, human L32 PCR primers, human lymphotoxin PCR primers, human MMP primers, human NOS PCR primers, human peptide hormone PCR primers, human phospholipase PCR primers, human PSA PCR primers, human receptor PCR primers, human small tandem repeat PCR primers, human structual protein PCR primers, human telomerase regulator PCR primers, human TIMP primers, human transcription factor PCR primers, human vimentin primers, and the like.

Non-limitative examples of mouse PCR primers include mouse 18S rRNA PCR primers, mouse apolipoprotein PCR primers, mouse apoptic gene PCR primers, mouse caspase PCR primers, mouse CD gene PCR primers, mouse chemokine primers, mouse chemokine receptor primers, mouse cycloxygenase PCR primers, mouse cytokine primers, mouse ECM PCR primers, mouse growth factor primers, mouse housekeeping gene primers, mouse insulin-like growth factor BP PCR primers, mouse MMP PCR primers, mouse NOS PCR primers, mouse oncogene PCR primers, mouse receptor PCR primers, mouse transcription factor PCR primers, and the like.

Examples of rat PCR primers include, but are not limited to rat apolipoprotein PCR primers, rat apoptic PCR primers, rat caspase PCR primers, rat cathepsin PCR primers, rat chemokine PCR primers, rat ECM PCR primers, rat growth factor PCR primers, rat housekeeping gene PCR primers, rat insulin-like growth factor BP PCR primers, rat interferon PCR primers, rat interleukin primers, rat NOS PCR primers, rat oncogene PCR primers, rat receptor PCR primers, rat rRNA PCR primers, rRat TNF PCR primers, and the like.

Suitable viral PCR primers include, but are not limited to adenovrius PCR primers, cytomegalovirus PCR primers, Epstein-Barr virus PCR primers, heptatitis viral primers, Herpes Simplex virus PCR primers, Herpes Type 6 PCR primers, HIV PCR primers, HTLV PCR primers, influenza virus gene PCR primers, papilloma virus PCR primers, para-influenza virus PCR primers, respiratory syncytial virus PCR primers, Varicella-Zoster virus PCR primers, and the like.

The hydrolysis of the probe 10 releases (as depicted by the lightening bolt) a probe fragment 16 containing the co-enzyme 14. The co-enzyme (co-factor (CF) 14) of the probe fragment 16 may then combine with and activate an apo-enzyme 20 immobilized on the surface of a working electrode 22. It is to be understood that the apo-enzyme 20 may also be present in the sample when the assay is homogeneous. The combination of the probe fragment 16 and the apo-enzyme 20 forms a holo-enzyme 24, which is capable of catalyzing a reaction that converts a predetermined substrate 26 in the sample to a product 28 plus free electrons. These free electrons may reduce a co-substrate (i.e. a reactant that is transiently associated with the enzyme and becomes a product(s) that cooperates chemically with another substrate regarding formation of another product(s), a non-limitative example of which is an oxidant) with a relatively high oxidation potential (such as, for example, oxygen to hydrogen peroxide) or they may reduce a mediator M₍ᵣ₎, which is subsequently re-oxidized M₍ₒ₎ by the working electrode 22 at a lower more selective potential.

The activation of the apo-enzyme 20 by the probe fragment 16 and the subsequent reaction involving the holo-enzyme 24 results in the formation of an electrochemically measurable enzymatic signal. It is to be understood that the electrochemical measurement of the enzymatic signal corresponds to a measurement of the target DNA 17.

The embodiment shown in FIG. 2 is a homogeneous assay system. In such a homogeneous system, the intact probe 10 is substantially inactive. After the DNA amplification process, the PCR mixture solution (the probe 10, probe fragment 16, the multiplied target DNA 17) will be mixed with the mediator (M) and/or a co-substrate, the apo-enzyme 20, nucleic acids, and any other desired ingredients/reagents for electrochemical detection, such as those described herein. In such a homogeneous system, the intact probe 10 may be desirably configured such that it has the tendency to not bind with the apo-enzyme 20, to not activate the holo-enzyme 24, and to not generate an electrochemical signal that is not representative of the DNA sample 17.

In a non-homogeneous assay system (see FIG. 4), the mediator (M) and the apo-enzyme 20 or holo-enzyme 24 may be coated on a working electrode 22 surface. It is to be understood that after the DNA amplification process, the PCR solution containing multiple copies of the target DNA (amplicon), the fragment probe 16, and the oligonucleotide probe 19 may be injected on the modified working electrode 22 surface for electrochemical detection.

Referring back to the DNA amplification processes, in an embodiment using an end-point detection DNA amplification process, holo-enzyme 24 activity (which generates the enzymatic signal) is measured before and after the entire process. The methods for detection include, but are not limited to voltammetry, amperometry, coulometry, potentiometry, conductivity, and the like under oxidative or reductive conditions. It is to be understood that a sample designation as to whether “positive (with target DNA)” or “negative (without target DNA)” for a given DNA sequence may depend, at least in part, on a predetermined criterion involving the magnitude of the change in electrochemical signal observed before and after the amplification process. In one embodiment using end-point detection, the PCR and detection processes may be performed sequentially as two separate steps in two separate and/or different spatial environments. In another embodiment using end-point detection, the PCR and electrochemical detection may be batch processed in one integrated step in the same spatial environment. In a non-limitative example using batch processing, thermophilic or thermally stabilized enzymes (non-limitative examples of which include sol-gel and probe encapsulated by biologically localized embedding (PEBBLE)) may be used.

In an embodiment using a real-time detection DNA amplification process, holo-enzyme 24 activity (which generates the enzymatic signal) is measured continuously, or in many closely spaced (in time) discrete measurements, throughout the entire process. The electrochemical signal may be detected using the methods previously described under oxidative or reductive conditions. It is to be understood that a sample designation as to whether “positive (with target DNA)” or “negative (without target DNA)” for a given DNA sequence may depend, at least in part, on a predetermined criterion involving the magnitude of the Delta comparing signal measurements before and after the PCR for each thermal cycle. In one embodiment using real-time detection, the PCR and detection processes may be performed sequentially as two separate steps in two separate and/or different spatial environments. In another embodiment using real-time detection, the PCR and electrochemical detection may be batch processed in one integrated step in the same spatial environment. In a non-limitative example using batch processing, thermophilic or thermally stabilized enzymes (non-limitative examples of which include sol-gel and PEBBLE) may be used.

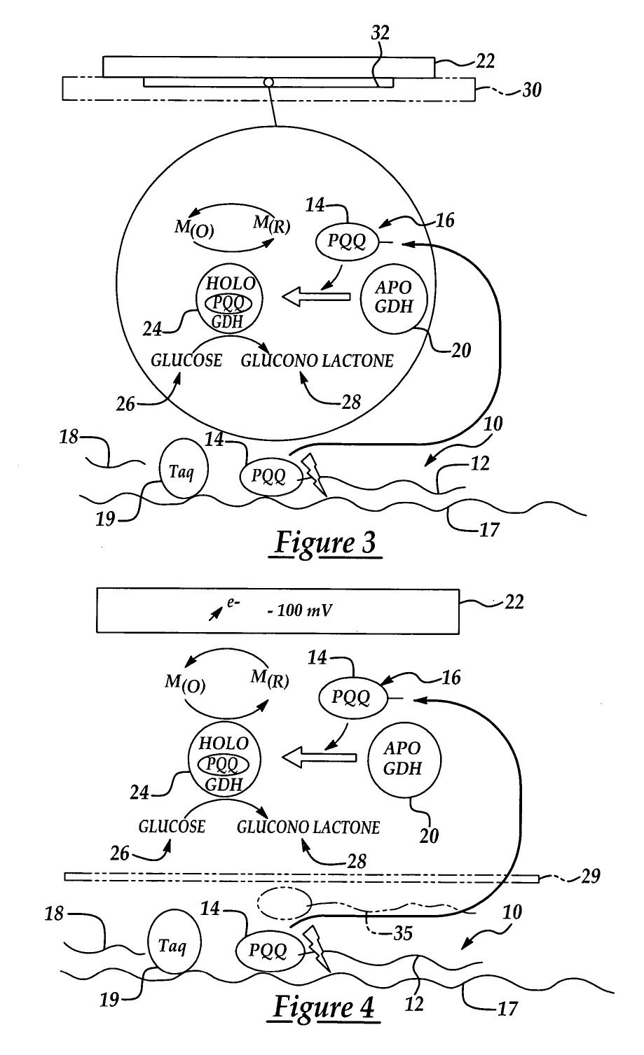

A specific non-limitative example of an embodiment of the method is depicted in FIG. 3. The intact probe 10 includes the sequence 12 having PQQ, as the co-factor (CF) 14, conjugated thereto. During the DNA amplification process, 5′→3′ exonuclease activity as a component of polymerase enzyme 19 (e.g. Taq DNA polymerase), results in the hydrolysis of the probe 10. A fragment oligo containing PQQ (i.e. probe fragment 16) is cleaved from the intact probe 10 during the DNA amplification process (PCR), thereby forming probe fragment 16, which combines with apo-enzyme 20 to form holo-enzyme 24.

As shown, the probe fragment 16 combines with apo-glucose dehydrogenase (apo-GDH) 20 to activate the apo-enzyme 20 and form the holo-GDH 24, which amplifies the enzymatic signal. In this non-limitative example, the substrate 26, glucose, is converted to a glucono lactone product 28 and free electrons.

It is to be understood that detecting the enzymatic signal may be accomplished, for example, using a two or three electrode voltammetric, amperometric, coulometric, conductometric, or potentiometric system. The system may include working electrode(s) 22, reference electrode(s), and counter electrode(s). In an embodiment the electrodes may be any suitable material, including, but not limited to platinum, platinum black, carbon, carbon paste, silver, silver paste, silver/silver chloride, silver/silver chloride paste, gold, gold paste, and/or the like, and/or mixtures thereof.

The working electrode(s) 22 may be modified to accomplish the electrochemical measurement of the enzymatic signal. Modifications to the working electrode 22 may be covalent or adsorptive in nature. For example, the working electrode 22 may be pretreated with electrochemically active mediators and/or modified with an enzyme, either or both of which may play a role in the signal transduction by the probe fragment 16 released from intact probe 10. It is to be understood that a mediator and/or an enzyme may be immobilized to the surface of the working electrode 22. The embodiment depicted in FIG. 3, and in particular the exploded portion, is an assay system in which the working electrode 22 is coated with a film 32 including a mediator (or co-substrate) and the apo-enzyme 20 (e.g. apo-GDH), which is physically separated from the solution containing the probe 10 and the sample 17 via an optional film 30.

In an alternate embodiment of the method (shown in FIG. 5), the working electrode 22 is coated (via physical adsorption or covalent bonding) with, or in solution with an active holo-enzyme 24. In this alternate embodiment, the electrochemical activity of the holo-enzyme 24 is transduced to the working electrode 22 via the probe fragment 16 having the mediator (M) 14′. This alternate embodiment will be discussed in further detail hereinbelow in reference to FIG. 5.

With the various embodiment(s) of the method described herein, there are two potential probes 10, 16 present during the DNA amplification process. The first probe is the intact probe 10 and the second probe is the probe fragment 16. It is to be understood that embodiment(s) of the method may advantageously substantially maximize the detection of the probe fragment 16 and substantially minimize the detection of the intact probe 10. Without being bound to any theory, it is believed that this may be accomplished by chemically designing the intact probe 10 such that it is inactive (such as in a homogeneous assay system, as depicted in FIG. 2) and fails, due in part to steric hindrance, to make contact with the apo- or holo-enzyme 20, 24.

Further, maximizing the detection of the probe fragment 16 may be accomplished by covering the working electrode 22 (modified or otherwise) with film 30. In the non-limitative example shown in FIG. 3, the apo-enzyme 20 adsorbed to a working electrode 22 may be covered with a polymeric nafion film 30. In one embodiment, the film 30 may be used to keep the reagents (e.g. apo-enzyme 20, mediator (M), co-substrate) close to the working electrode 22. In another embodiment, the film 30 is configured to sequester the intact probe 10 from the apo-enzyme 20. The film 30 may exhibit size or charge exclusion properties, thereby allowing diffusion of the small probe fragment 16, while substantially prohibiting diffusion of the intact probe 10 (for example, of a negatively charged intact probe 10).

In the embodiment depicted in FIG. 4, the assay system is non-homogeneous, in that the apo-enzyme 20 is separated from the active or potentially active intact probe 10. In this embodiment, it is to be understood that the sample including the target DNA 17 and the intact probe 10 may be a separate homogeneous solution. It is to be further understood that in such an embodiment, the active/potentially active probe 10 may undesirably have the tendency to bind with the apo-enzyme 20, to activate the holo-enzyme 24, and to generate an electrochemical signal that is not representative of the DNA sample 17.

Where the intact probe 10 is active/potentially active, various elements may be employed to keep the active intact probe 10 in the system and allow the fragment probe 16 having the co-factor (CF) 14 to release and react with the apo-enzyme 20. The previously described film 30 may be added to the surface of the electrode 22.

In an alternate embodiment, as also depicted in FIG. 4, additional optional reagent(s) 35 may be added to the sample or coated on the working electrode 22, prior to taking the electrochemical measurements. The reagent(s) 35 may complex the active/potentially active intact probe 10, thereby substantially reducing a background signal that may result from the presence of the probe 10. In a non-limitative example, the reagent 35 is a polycation, which may “tie up” a polyanion probe 10. In another non-limitative example, the reagent 35 is protamine or poly-L-lysine. The reagent 35 substantially prevents the reconstitution of the intact probe 10 with apo-enzyme 20 while allowing the binding of the probe fragment 16 with the apo-enzyme 20. The reagent 35 may bind to the probe 10 and form an aggregation such that the probe 10 may not combine with the apo-enzyme 20 (shown in FIG. 4) or holo-enzyme 24 (shown in FIG. 6), thereby substantially eliminating amplification of an enzymatic signal generated from the active/potentially active intact probe 10.

In a further embodiment depicted in FIG. 4, a barrier membrane 29 may be added between the probe 10 and the working electrode 22. The barrier membrane 29 allows the probe fragment 16 to diffuse through to the apo-enzyme 20 while substantially preventing diffusion of the intact probe 10. It is to be understood that barrier membrane 29 may be used in addition to reagent 35 or in place of reagent 35. Still further, it is to be understood that the reagent 35 and/or the barrier membrane 29 may be used in any system (e.g. the system depicted in FIG. 2) as disclosed herein, and is not limited to the system shown in FIG. 4.

Referring now to FIG. 5, an alternate embodiment of the method of detecting DNA 17 is depicted. In this non-limitative example embodiment, the probe 10 has a mediator (M) 14′ (a non-limitative example of which include ferrocene, labeled Fc in FIG. 6) conjugated to the sequence 12. Further, a holo-enzyme 24 is immobilized on the surface of the working electrode 22 or is present homogeneously in the sample/reaction mixture phase.

It is to be understood that in this embodiment, the enzyme activity may be fully functional prior to cleaving the probe fragment 16, as the holo-enzyme 24 is present from the beginning of the method. However, it is to be further understood that the electrons generated prior to the release of the probe fragment 16 may be used to form relatively stable reductant, and at such low oxidation potential, current may not be detectable. The mediator (M) 14′ may be electrochemically active and thus may serve as an electron transfer mediator from the holo-enzyme 24 to working electrode 22. The liberated probe fragment 16 (mediator (M) 14′) contacts the holo-enzyme 24, thereby increasing the conversion of substrate 26 to product 28 and releasing additional free electrons that are electrochemically measurable. It is to be further understood that the conversion of the substrate 26 assists in oxidizing the mediator (M) 14′ of the probe fragment 16, and that the oxidized mediator (M_{(Ox)}) will be reduced (M_{(R)}) by working electrode 22. As such, the activation of the holo-enzyme 24 continues until the depletion of the substrate 26.

FIG. 6 is a specific embodiment of the method depicted in FIG. 5. The mediator (M) 14′ is ferrocene (Fc). The probe fragment 16 having the Fc mediator (M) 14′ reacts with the holo-enzyme 24, such as peroxidase (POX), to increase (catalyze) the conversion of substrate 26 (hydrogen peroxide) to product 28 (water) through the effective electron transfer between holo-enzyme 24 and working electrode 22 by mediator (M) 14′ (i.e. the continuous cycling conversion of Fc_{(R)} to Fc_{(Ox)} by holo-enzyme 24 and then Fc_{(Ox)} to FC_{(R)} by working electrode 22).

FIG. 6 also depicts a sodium dodecyl sulfate (SDS) coating 34 established on the working electrode 22. With this coating 34, the surface of the working electrode 22 has an anionic surfactant thereon. It is to be understood that the electrode 22 may be pretreated with such a coating 34 in order to substantially increase the accumulation of the mediator (M) 14′ on the surface of working electrode 22.

It is to be understood that the embodiments depicted in FIGS. 5 and 6 may include an active/potentially active intact probe 10 with mediator 14′ that is capable of having significant activity with the holo-enzyme 24, thereby resulting in an enzymatic signal that is not indicative of the target DNA sample 17. In such an embodiment, reagent(s) 35 (described above in reference to FIG. 4) and/or barrier membrane(s) 29 (described above in reference to FIG. 4) may be included in the system in order to substantially suppress or eliminate the undesirable signal from the intact probe 10.

An embodiment of the probe 10 may be incorporated into a portable diagnostic device 100. In a non-limitative example using end-point PCR DNA amplification, the device 100 is a glucometer-type two or three electrode sensing system. In a non-limitative example using real-time PCR DNA amplification, the device 100 is a microfluidic or a macrofluidic platform having a sample concentrator and/or cell lyses components.

Experimental

Sequestration of apo-Glucose Dehydrogenase (GDH) to a Carbon Electrode With a Nafion® Film Thereon.

The working carbon electrode was first preloaded with 0.4 μl of APO-GDH (1 mg/ml). After drying, the preloaded working carbon electrode was covered with a drop of 0.5 μl of Nafion® solution (0.5%) to form a film on the electrode surface. The droplet was allowed to dry.

Development of Electrochemical Assay for PQQ with GDH with Pt-Black Electrode.

A platinum wire, having a 0.5-mm diameter, was sealed in glass (3-mm diameter) and was platinized by the procedure previously described by Y. Lee et al. in Analytical Chemistry (2004). The platinum wire formed the basis of the APO-GDH mounted sensor. 4 μL of APO-GDH solution (1 mg/ml) was loaded on the surface of the platinized platinum disk electrode and dried at room temperature. The APO-GDH coated electrode was covered with a dialysis membrane (molecular weight cut off (MWCO)=12 kDa, cellulose acetate) and held in position using an O-ring.

In amperometric APO-GDH sensors, current passed, due to the electrochemical oxidation of phenazoinum methosulfate (PMS), was detected as an output signal by applying a small oxidative potential. The solution was stirred during detection of the measurements. APO-GDH sensors (without PQQ) were tested in terms of their responses to the addition of different levels of PQQ into the phosphate buffered saline (PBS) solution containing 20 mM glucose and 2 mM CaCl₂.

Electrochemical Assay for PQQ with GDH with Carbon Strip Electrode.

To increase the sensitivity of a PQQ assay, the working carbon electrode was first preloaded with 0.4 μl, APO-GDH (1 mg/ml). After drying, the electrode was covered with a drop of 0.2 μl, Nafion® solution (0.5%). The droplet was allowed to dry. The working electrode was preloaded and incubated for 1 minute with 10 mM PBS buffer (pH 7.0, total volume 50 mL). 1 μl CaCl₂, 1 μl glucose, 2.5 μl PMS, and 1 μl PQQ were added to the working electrode.

Chronoamperometry was used to measure the activity of GDH following the addition of PQQ. A constant potential was maintained at a small oxidative potential during measurements. PQQ at 100 pM was detectable.

Electrochemical Assay for PQQ Probe (in PCR Sample) with GDH with Carbon Strip Electrode.

To increase the sensitivity of a PQQ probe assay, the working carbon electrode was first preloaded with 0.2 μl PMS and then 0.2 μl APO-GDH (1 mg/ml). After drying, the electrode was spin coated with a drop of 0.2 μl Nafion® solution (0.5%). The working carbon electrode was preloaded and incubated for 1 minute with PCR samples (substrates, enzyme activity enhancers included in PCR mixture solution). 1 μl PMS was added before the measurements were taken.

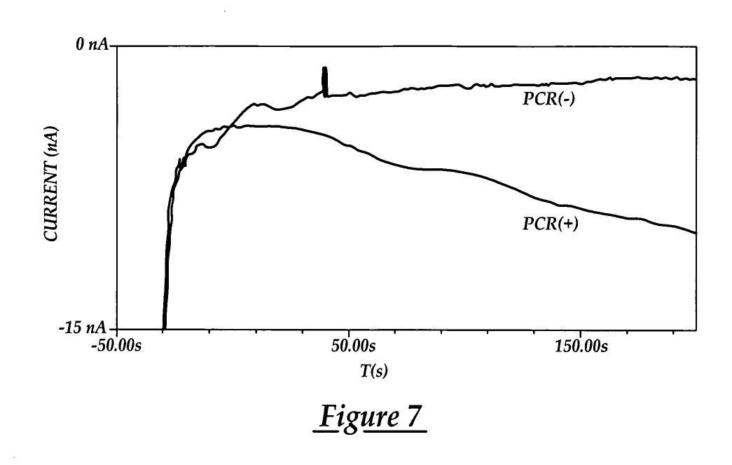

FIG. 7 is a graph depicting an amplified amperometric signal involving glucose dehydrogenase (GDH) activity when a positive and a negative PCR sample were tested. The PCR sample contained a PQQ labeled oligonucleotide probe for group beta streptococcus (GBS) gene targeting. The PCR samples were collected after 50 thermal cycles.

The PCR conditions were as follows. The mixture included 400 nM Forward Primer, 400 nM Reverse Primer, 4 mM MgCl₂, 500 μg/ml BSA, 200 μM dNTPs, 50 mM KCl, 10 mM Tris pH 8.3, 200 nM PQQ probe, and 1000 copies of genomic GBS. The thermal cycling conditions included a melting temperature of 95° C. and a primer extension temperature of 60° C.

Development of an Electrochemical Assay for Ferrocene with Peroxidase.

The oxidation of ferrocene to ferrocenyl and its subsequent reduction back to ferrocene may be detected electrochemically. The PCR assay included a ferrocene e-probe and the enzymatic assay involved an active holo-POX that is detected electrochemically with the release of free ferrocene.

The working carbon electrode was pretreated with a drop of SDS (10%) just large enough to cover the electrode surface. The droplet was allowed to dry and the electrode was rinsed in distilled water. This treatment resulted in the application of a thin layer of anionic surfactant to the carbon electrode surface. The addition of this layer resulted in a more extensive accumulation of dimethylamino (DMA) ferrocene (or probe fragment of the Fc probe) over time. The working carbon electrode was preloaded and incubated for 10 minutes with 1 μl Fc (or Fc Probe). 8 μl POX and 1 μl hydrogen peroxide were also added.

Chronoamperometry was used to measure the activity of POX following the addition of hydrogen peroxide. A constant potential was maintained at a small reductive potential when hydrogen peroxide was added.

While several embodiments have been described in detail, it will be apparent to those skilled in the art that the disclosed embodiments may be modified. Therefore, the foregoing description is to be considered exemplary rather than limiting.

What is claimed:

1. An oligonucleotide probe for enzyme amplified electrochemical target DNA detection, the oligonucleotide probe comprising:
a site-specific sequence; and
a co-factor conjugated to the site-specific sequence or a mediator conjugated to the site-specific sequence;
wherein the co-factor is adapted to produce an enzymatic signal that is electrochemically detectable and the mediator is adapted to enhance an enzymatic signal that is electrochemically detectable.

2. The oligonucleotide probe as defined in claim 1 wherein the co-factor comprises one of a prosthetic group, a metal-ion activator, and a do-enzyme.

3. The oligonucleotide probe as defined in claim 1 wherein the co-factor is one of pyrroloquinoline quinone, flavin adenine dinucleotide, nicotinamide adenine dinucleotide phosphate, and heme.

4. The oligonucleotide probe as defined in claim 1 wherein the mediator is one of ferrocene, ferrocene derivatives, dichlorophenol indolphenol, ubiquinone, Ru(III) complexes, Os(III) complexes, and phenazoinum methosulfate.

5. The oligonucleotide probe as defined in claim 1 wherein the site specific sequence is 5′-amino-hexyl-AGAAGTACATGCTGATCAAGTGACAACTCCACA-3′, the prosthetic group is pyrroloquinoline quinone, and the target DNA is from group beta streptococcus.

6. The oligonucleotide probe as defined in claim 1 wherein the enzymatic signal is electrochemically detectable via voltammetry, amperometry, coulometry, potentiometry, conductivity, and combinations thereof.

7. The oligonucleotide probe as defined in claim 1 wherein the co-factor is adapted to release from the site-specific sequence and to bind to and activate an apo-enzyme, thereby forming a holo-enzyme that is adapted to catalyze a reaction to produce electrons.

8. The oligonucleotide probe as defined in claim 1 wherein the mediator is adapted to release from the site-specific sequence and to react with a holo-enzyme.

9. A method of detecting target DNA in a sample, the method comprising:
performing a DNA amplification process on the sample;
exposing an oligonucleotide probe to exonuclease activity, the oligonucleotide probe including a site-specific sequence and a co-factor conjugated to the site-specific sequence or a mediator conjugated to the site-specific sequence, the exposing thereby releasing a probe fragment including the co-factor or the mediator;
combining the probe fragment with an apo-enzyme or a holo-enzyme, wherein combining the probe fragment having co-factor with the apo-enzyme produces an enzymatic signal that is electrochemically detectable or wherein combining the probe fragment having the mediator with the holo-enzyme enhances an enzymatic signal that is electrochemically detectable; and
electrochemically detecting the enzymatic signal, thereby detecting the target DNA.

10. The method as defined in claim 9 wherein the DNA amplification process is at least one of real time PCR, end-point PCR, and RCA.

11. The method as defined in claim 10 wherein the DNA amplification process is real time PCR and wherein the enzymatic signal is detected at predetermined intervals during the amplification process.

12. The method as defined in claim 10 wherein the DNA amplification process is end-point PCR and wherein the enzymatic signal is detected prior to and after the amplification process.

13. The method as defined in claim 10 wherein the DNA amplification process includes exposing the sample to a PCR mixture including at least one of substrates, co-substrates, buffers, mediators, stabilizers, and mixtures thereof.

14. The method as defined in claim 9 wherein the performing, exposing, combining, and detecting occur one of substantially simultaneously and sequentially.

15. The method as defined in claim 9 wherein the apo-enzyme or the holo-enzyme is immobilized on a surface of an electrode.

16. The method as defined in claim 15 wherein the electrode comprises a film layer covering the apo-enzyme or the holo-enzyme.

17. The method as defined in claim 15, further comprising establishing a barrier membrane between the oligonucleotide probe and the electrode, the barrier membrane adapted to diffuse the probe fragment and prohibit diffusion of the oligonucleotide probe.

18. The method as defined in claim 9 wherein electrochemically detecting the enzymatic signal may be accomplished via voltammetry, amperometry, coulometry, potentiometry, conductivity, and combinations thereof.

19. The method as defined in claim 9 wherein the probe fragment having the co-factor combines with the apo-enzyme, and the method further comprises:
binding the co-factor with the apo-enzyme, thereby activating the apo-enzyme and forming a holo-enzyme; and
converting a substrate in the sample to a product and free electrons via a reaction involving the holo-enzyme as a catalyst.

20. The method as defined in claim 9 wherein the holo-enzyme produces an enzymatic signal prior to being combined with the probe fragment having the mediator, and wherein the method further comprises mediating electron transfer between the holo-enzyme and an electrode, thereby enhancing the enzymatic signal of the holo-enzyme such that the signal is detectable electrochemically.

21. The method as defined in claim 9, further comprising adding a reagent to the sample after performing the DNA amplification process and prior to detecting the enzymatic signal, thereby complexing the oligonucleotide probe and prohibiting its binding with the apo-enzyme.

22. The method as defined in claim 21 wherein the reagent is one of protamine and poly-L-lysine.

23. The method as defined in claim 9, further comprising adding a reagent to the sample, thereby complexing the oligonucleotide probe and prohibiting its combining with the holo-enzyme.

24. The method as defined in claim 9 wherein the co-factor comprises one of a prosthetic group, a co-enzyme, and a metal-ion activator.

25. The method as defined in claim 24 wherein the co-factor is one of pyrroloquinoline quinone, flavin adenine dinucleotide, nicotinamide adenine dinucleotide phosphate, and heme.

26. The method as defined in claim 9 wherein the mediator is one of ferrocene, ferrocene derivatives, dichlorophenol indolphenol, ubiquinone (Qo), Ru(III) complexes, Os(III) complexes, and phenazoinum methosulfate.

27. The method as defined in claim 9 wherein the site-specific sequence is 5′-amino-hexyl-AGAAGTACATGCTGATCAAGTGACAACTCCACA-3′, the prosthetic group is pyrroloquinoline quinone, and the target DNA is from group beta streptococcus.

28. The method as defined in claim 9 wherein the apo-enzyme or holo-enzyme, the target DNA, and the oligonucleotide probe are mixed in a homogeneous assay system.

29. The method as defined in claim 9 wherein the mediator and the apo-enzyme or holo-enzyme coated on an electrode surface in a heterogeneous assay system and wherein a PCR solution containing multiple copies of target DNA, the fragment probe, and the oligonucleotide probe is injected on a modified electrode surface for electrochemical detection.

30. A diagnostic device for detecting target DNA, comprising:
at least one electrode having an apo-enzyme or a holo-enzyme immobilized thereon; and
an oligonucleotide probe in electrochemical contact with the at least one electrode, the probe including:
a site-specific sequence; and
one of a co-factor conjugated to the site-specific sequence and a mediator conjugated to the site-specific sequence;
wherein the co-factor or the mediator is adapted to release from the probe to form a probe fragment;
wherein the probe fragment having the co-factor binds to the apo-enzyme to produce an enzymatic signal that is electrochemically detectable;
or wherein the probe fragment having the mediator reacts with the holo-enzyme to enhance an enzymatic signal that is electrochemically detectable.

31. The diagnostic device as defined in claim 30 wherein the co-factor comprises one of a prosthetic group, a co-enzyme, and a metal-ion activator.

32. The diagnostic device as defined in claim 30 wherein the co-factor is one of pyrroloquinoline quinone, flavin adenine dinucleotide, nicotinamide adenine dinucleotide phosphate, and heme.

33. The diagnostic device as defined in claim 30 wherein the mediator is one of ferrocene, ferrocene derivatives, dichlorophenol indolphenol, ubiquinone (Qo), Ru(III) complexes, Os(III) complexes, and phenazoinum methosulfate.

34. The diagnostic device as defined in claim 30 wherein the site-specific sequence is 5′-amino-AGAAGTACATGCTGATCAAGTGACAACTCCACA-3′, the co-factor is pyrroloquinoline quinone, and the target DNA is from group beta streptococcus.

35. The diagnostic device as defined in claim 30 wherein the enzymatic signal is electrochemically detectable via voltammetry, amperometry, coulometry, potentiometry, conductivity, and combinations thereof.

36. The diagnostic device as defined in claim 30 wherein the at least one electrode comprises a film layer covering the apo-enzyme or the holo-enzyme, the film layer exhibiting at least one of charge and size exclusion properties to allow the probe fragment to contact the apo-enzyme or the holo-enzyme.

37. The diagnostic device as defined in claim 30, further comprising a barrier membrane established between the oligonucleotide probe and the electrode, the barrier membrane adapted to diffuse the probe fragment and prohibit diffusion of the oligonucleotide probe.
